(19) United States
(12) Patent Application Publication
(10) Pub. No.: US 2019/0290176 A1
(43) Pub. Date: Sep. 26, 2019

(54) Systems And Methods For Determining Physiological Information With A Computing Device

(71) Applicant: The Regents of the University of Colorado, a body corporate, Denver, CO (US)

(72) Inventors: Tam VU, Boulder, CO (US); Robin DETERDING, Boulder, CO (US); Anh NGUYEN, Denver, CO (US); Phuc NGUYEN, Denver, CO (US)

(73) Assignee: The Regents of the University of Colorado, a body corporate, Denver, CO (US)

(21) Appl. No.: 16/342,944

(22) PCT Filed: Oct. 19, 2017
(86) PCT No.: PCT/US17/57465

§ 371 Date: Apr. 17, 2019

Related U.S. Application Data
(60) Provisional application No. 62/410,299, filed on Oct. 19, 2016.

(51) Int. Cl. A61B 5/1455; A61B 5/00

(52) U.S. Cl. CPC A61B 5/14552; A61B 2562/0233; A61B 5/0033; A61B 5/0077

(57) ABSTRACT

A physiological information detection system may include an add-on device in electronic communication with a computing device. The add-on device includes a body and a first light filter retained in the body. The first light filter filters light emitted by the light source of the computing device. A lens is also retained in the body proximate the first light filter. The lens directs light exiting the first light filter out a surface of the first lens. The body retains a second lens that receives into a surface of the second lens light exiting the first lens. The surface of the first lens and the surface of the second lens are substantially coplanar. A second light filter is also retained in the body proximate the second lens. The sensor detects the light exiting the second light filter.

### FIELD

This disclosure relates to systems and methods for measuring physiological information using a light source and image sensor of a computing device.

### BACKGROUND

Many people suffering from cardio pulmonary diseases monitor blood vitals to track their condition. For example, acute asthma has been spreading rapidly in recent years. Oxygen levels in the blood, for example, may be indicative of underlying conditions. Pulse oximetry is a non-invasive method to measure peripheral oxygen saturation (SpO₂). SpO₂ approximates arterial oxygen saturation (SaO₂) well enough to be a convenient and relatively accurate way to measure oxygen saturation. However, portable pulse oximetry devices tend to be relatively costly. The measurements from standalone portable pulse oximetry devices can also be erratic in some instances. Some portable pulse oximetry devices also rely on clamping interfaces that may be too small for pediatric applications.

Also, many people may benefit from improved access to their physiological information. Conventional systems, devices, and methods for detecting and/or measuring physiological information, such as oxygen, glucose, and/or urea levels, are not readily available to individual consumers.

### SUMMARY

Systems and methods for measuring physiological information and/or physiological parameters, such as oxygen saturation in blood, are disclosed herein. According to various embodiments, the systems include a computing device and an add-on device in electronic communication with the computing device. The computing device, according to various embodiments, includes a light source and an image sensor. In various embodiments, the add-on device includes a body and a first light filter retained in the body. The first light filter is configured to filter light emitted by the light source of the computing device, according to various embodiments. A lens may also be retained in the body proximate the first light filter. The lens directs light exiting the first light filter out a surface of the first lens, according to various embodiments. A second lens may be retained in the body and may receive light exiting the first lens via a surface of the second lens. In various embodiments, the surface of the first lens and the surface of the second lens are substantially coplanar. A second light filter may also be retained in the body proximate the second lens. The sensor is configured to detect the light exiting the second light filter, according to various embodiments.

Also disclosed herein, according to various embodiments, are methods for measuring physiological information and/or parameters, such as oxygen saturation in blood, that include the step of calculating a first mean value of light intensity detected by a first partition of an image sensor on the computing device at a first time for a red wavelength to generate a red Photoplethysmogram (PPG) signal. The method also includes calculating a second mean value of light intensity detected by a second partition of the image sensor on the computing device at the first time for an infrared wavelength to generate an infrared PPG signal, according to various embodiments. The red PPG signal and the infrared PPG signal may be filtered to allow frequencies ranging from 0.5 Hz to 4 Hz to pass. The method further includes calculating the absorptivity ratio from the filtered red PPG signal and the filtered infrared PPG signal, and calculating the oxygen levels based on the absorptivity ratio, according to various embodiments.

In various embodiments, the physiological information device includes a camera covering portion. The camera covering portion may include the second lens and the second filter. In various embodiments, the camera covering portion includes the second light filter and a third light filter. In various embodiments, the second light filter is a red filter and the third light filter is a green filter. In various embodiments, the device includes a light covering portion, wherein the light covering portion comprises the first light filter and the first lens. In various embodiments, the light covering portion comprises a plurality of apertures that limit an amount of light energy from the light source. In various embodiments, the body of the add-on physiological information device comprises a flexible material and a rigid material. The flexible material may be configured to absorb/damp vibrational movement.

The foregoing features and elements may be combined in various combinations without exclusivity, unless expressly indicated herein otherwise. These features and elements as well as the operation of the disclosed embodiments will become more apparent in light of the following description and accompanying drawings.

### BRIEF DESCRIPTION

The subject matter of the present disclosure is particularly pointed out and distinctly claimed in the concluding portion of the specification. A more complete understanding of the present disclosure, however, may be obtained by referring to the detailed description and claims when considered in connection with the drawing figures, wherein like numerals denote like elements.

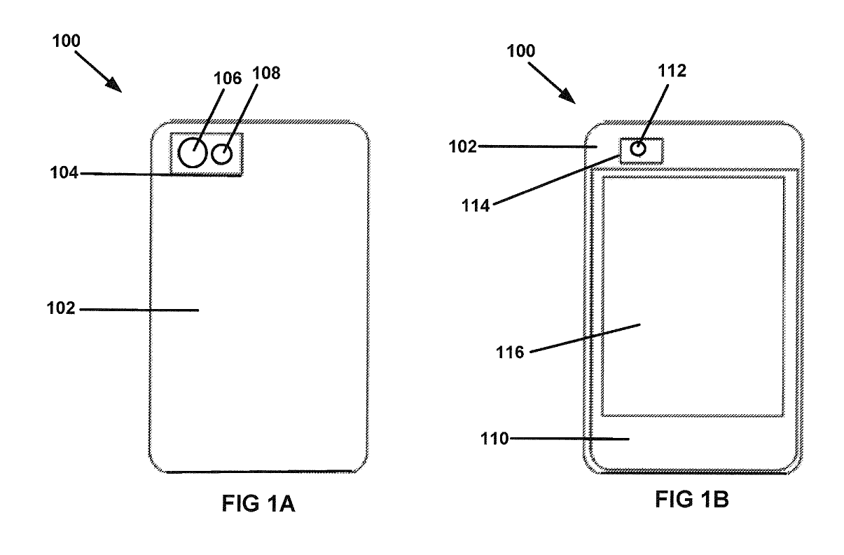

FIGS. 1A and 1B illustrate an example of a portable device with an add-on device configured to determine/measure physiological information, such as oxygen saturation and heart rate, in accordance with various embodiments;

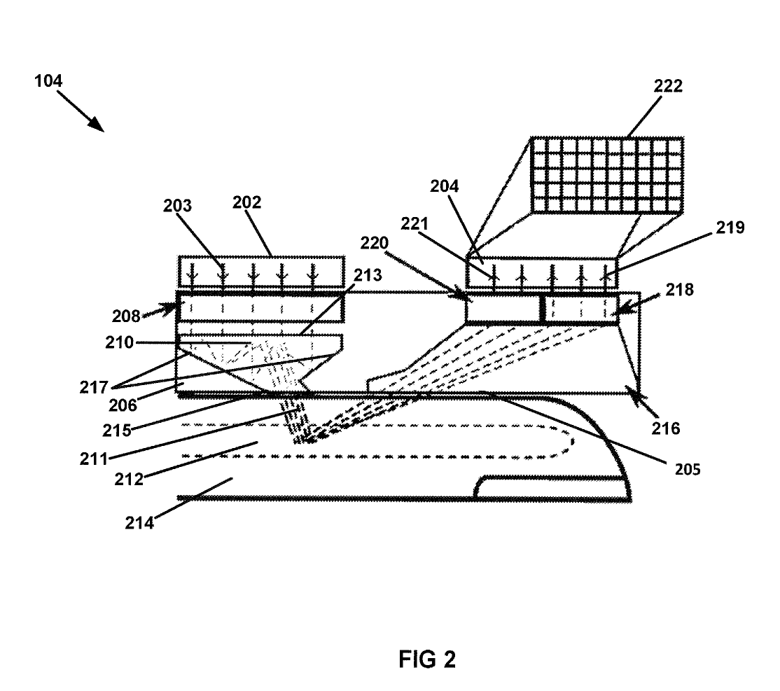

FIG. 2 illustrates an example of an add-on device for a portable device configured to determine/measure physiological information, such as oxygen saturation and heart rate, by filtering light into the desired wavelengths and capturing an image using a digital camera integrated into the portable device, in accordance with various embodiments;

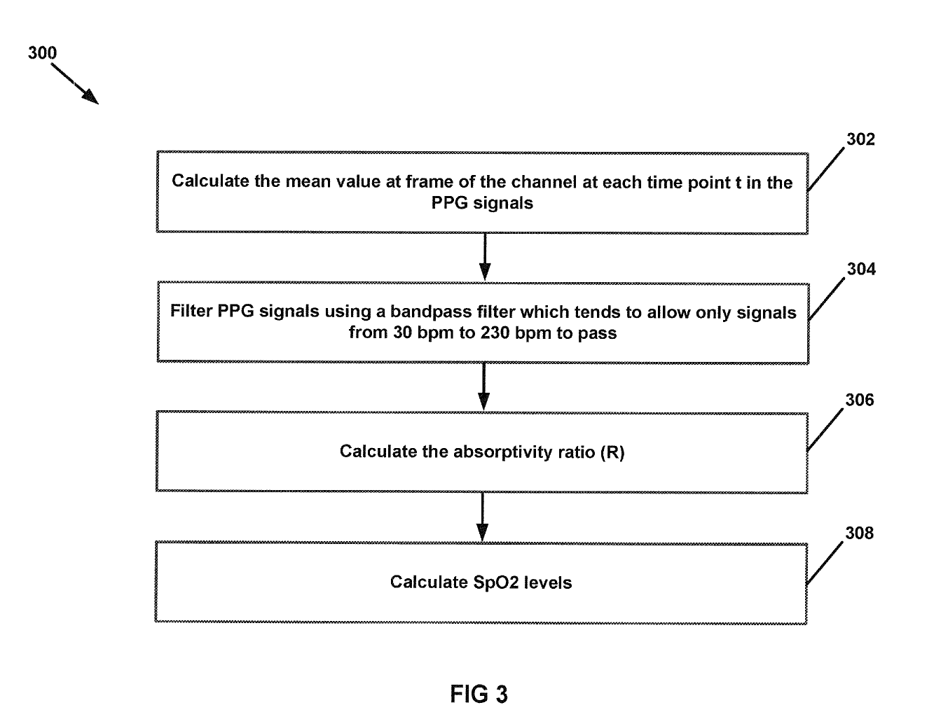

FIG. 3 illustrates a process for detecting physiological information, such as oxygen saturation, using an add-on device for a portable device, in accordance with various embodiments;

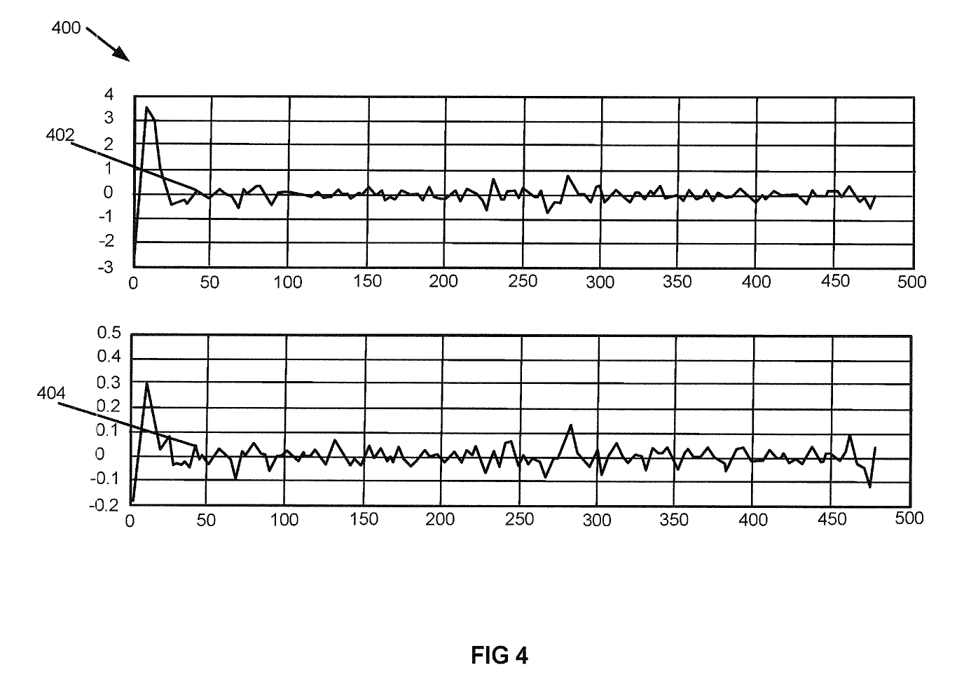

FIG. 4 illustrates captured signals for intensity versus time at two different light wavelengths, in accordance with various embodiments; and

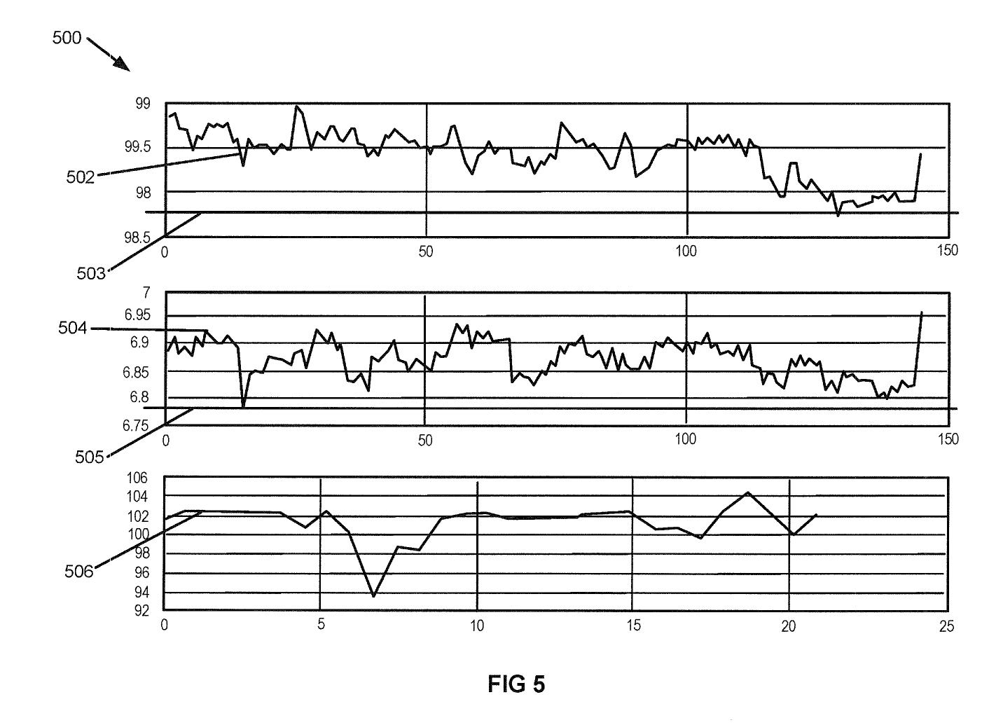

FIG. 5 illustrates signal analysis of two intensity signals and the resulting SpO₂ estimation versus time, in accordance with various embodiments.

### DETAILED DESCRIPTION

The detailed description of various embodiments refers to the accompanying drawings, which show various embodiments by way of illustration. While these various embodiments are described in sufficient detail to enable those skilled in the art to practice the disclosure, it should be understood that other embodiments may be realized and that logical and mechanical changes may be made without departing from the spirit and scope of the disclosure. Thus, the detailed description is presented for purposes of illustration only and not of limitation. For example, the steps recited in any of the method or process descriptions may be executed in any order and are not limited to the order presented. Moreover, any of the functions or steps may be outsourced to or performed by one or more third parties. Furthermore, any reference to singular includes plural embodiments, and any reference to more than one component may include a singular embodiment.

Disclosed herein, according to various embodiments, are devices, systems, and methods for determining and/or measuring physiological information/factors, such as oxygen saturation in the blood stream of a user. For example, the disclosed devices, systems, and methods may be implemented as an oxygen saturation measuring system and/or may be implemented to determine the levels of various compounds in the body or bloodstream of a user, such as glucose, urea, etc. Thus, while numerous details are included herein pertaining to oxygen saturation measurements, the scope of the present disclosure is not limited to oxygen saturation detection and the principles and features of the disclosed devices, systems, and methods may be utilized in various physiological detection implementations.

Generally, the physiological information detection system provided herein includes a portable device and an add-on device. The add-on device is configured to be in electronic communication with the portable device. The add-on may generate light or may redirect light from the portable device to shine into a finger or other body part of a user, and said light may be reflected into the camera of the portable device. In various embodiments, the light is filtered into predetermined wavelengths that result in accurate readings of physiological information, such as oxygen saturation levels, and is directed to partitioned portions of a camera sensor. Measurements taken using light with infrared and red wavelengths have improved accuracy versus measurements taken using the entire spectrum of electromagnetic radiation emitted by the flash on the portable device or other light source of the add-on device, according to various embodiments.

With reference to FIGS. 1A and 1B, an exemplary physiological information system 100 is shown, in accordance with various embodiments. the physiological information system 100 may be an SpO₂ system. As mentioned above, the system 100 comprises a computing device 110 and an add-on device 104, according to various embodiments. Add-on device 104 may be integrated into a case 102 for ease of use and carrying. Add-on device 104 may also be packaged independent of a case 102. For example, the add-on device 104 may be configured to snap on to a portion of the computing device 110. Add-on device 104 is capable of electronic communication with computing device 110 over a wireless channel (e.g., a Bluetooth channel or an IEEE 802.11 wireless communication channel) and/or a wired channel (e.g., a USB, micro-USB, lightning port, audio jack, or other communication port in computing device 110).

Computing device 110 may include one or more cameras and/or flashes. FIG. 1A depicts the rear of computing device 110, rear-facing camera 106 and rear-facing flash 108. FIG. 1B illustrates the front of computing device 110 having a front-facing camera 112. Front panel 114 of add-on device 104 may optically connect rear-facing flash 108 to front panel 114 such that light from rear-facing flash 108 is delivered to front panel 114. Front panel 114 may also include a separate light source such as an LED light source. Add-on device 104 and/or front panel 114 may make SpO₂ measurements based on the light absorbed into rear-facing camera 106 and/or front-facing camera 112, respectively.

For example, the computing device 110 may take the form of a computer or processor, or a set of computers/processors, although other types of computing units or systems may be used. Exemplary computing devices 110 include smartphones, tablets, laptops, notebooks, hand held computers, personal digital assistants, cellular phones, smart phones (e.g., iPhone®, BlackBerry®, Android®, etc.), wearables (e.g., smart watches and smart glasses), Internet of things (IOT) devices or any other device capable of generating light and/or capturing red and infrared light. Although red and IR light are identified, other light channels may be used in the present device by using the corresponding light filters as described below. Each computing device 110 may run an application to interpret the red and infrared signals arriving at front-facing camera 112 and/or rear-facing camera 106. The application may use screen 116 to output results and receive input from a user.

Referring now to FIG. 2, an exemplary add-on device 104 is shown for measuring physiological information, such as SpO₂, in accordance with various embodiments. Although FIG. 1A depicts add-on device 104 as being on a rear-facing side of computing device 110, add-on device 104 may be located anywhere on computing device 110 proximate a camera and/or light source. Add-on device 104 is in electromagnetic communication with light source 202 (e.g., of rear-facing flash 108 in FIG. 1A). Add-on device 104 includes a body 206. Body 206 may comprise a molded plastic, thermoset plastic, metal, rubber, or other material. In that regard, body 206 may be a protective case similar to case 102 of FIGS. 1A and 1B. Body 206 may retain filter 208 and lens 210 in position relative to one another.

Light 203 emitted from light source 202 passes through filter 208. Filter 208 removes electromagnetic radiation of undesired wavelengths. For example, filter 208 of add-on device 104 filters light 203 so that all visible light aside from red light is removed. Filter 208 may also filter ultraviolet light from light 203. Filter 208 may thus allow light from the infrared light and red light to pass through. The wavelength of infrared light ranges from about 800 nm to about 1 mm. The wavelength of red light begins at the border of infrared light and is generally about 650 nm. Red light has a wavelength ranging from about 800 nm to about 600 nm.

Light 203 passing through filter 208 enters lens 210 through surface 213. Lens 210 is a transparent or translucent material with a geometry and refractory index suitable to direct light 211 out of lens 210 at surface 215 and towards blood-rich portion 212 of body part 214. Body part 214 may be, for example, a finger, a hand, a forearm, an ear, or a neck. Light 211 may be filtered light containing electromagnetic radiation in the frequencies selected by filter 208. Lens 210 may be made of glass, polycarbonate, plastic, or other suitable lens material. Surfaces of lens 210 where light escaping is undesirable, such as surfaces 217, may be coated with a reflective material or a material of a different refractory index than lens 210 to reflect or otherwise alter the path of light 203 within lens 210. Lens 210 may thus tend to focus and direct light 211 towards body part 214 at an angle. The angle at which light 211 approaches body part 214 may be selected based on the distance between light source 202 and sensor 204 (of rear-facing camera 106, for example) so that suitable light levels reflect from body part 214 generally towards sensor 204. The angle at which light 211 approaches body part 214 may also be selected based on the distance between light source 202 or sensor 204 and body part 214 so that suitable light levels reflect from body part 214 generally towards sensor 204. Light 211 may thus travel in a u-shaped or v-shaped path from emission at the light source 202, into body part 214, and back to sensor 204.

Light 211 reflecting from blood-rich portion 212 of body part 214 enters lens 216. Body 206 may retain lens 216 relative to lens 210. Light 211 may enter lens 216 at surface 205. Surface 205 and surface 215 may be substantially coplanar such that body part 214 lays across and/or covers surface 215 as well as surface 205. Lens 216 may tend to guide light 211 towards infrared filter 218 and red filter 220. Body 206 may retain infrared filter 218 and red filter 220, which are described in greater detail below, relative to other components of add-on device 104. Lens 216 may be treated in a manner similar to lens 210 as described above to facilitate the transfer of infrared and red light components of light 211 towards infrared filter 218 and red filter 220.

Infrared filter 218 is a translucent or transparent material configured to remove wavelengths other than infrared from light 211. For example, infrared filter 218 may be configured to allow infrared light to pass and remove red light. Similarly, red filter 220 is a translucent or transparent material configured to remove wavelengths other than red from light 211. For example, red filter 220 may be configured to allow red light to pass and remove infrared light. The refractory index of infrared filter 218 and/or red filter 220 may be selected to direct light 211 exiting lens 216 towards sensor 204. Infrared light 219 may pass through infrared filter 218 towards a partition of sensor 204. Red light 221 may pass through red filter 220 towards a partition of sensor 204.

Sensor 204 may include partitions 222 with red light and infrared light captured at different partitions 222 of sensor 204. Sensor 204 may include partitions 222 that receive both red and infrared light. The partitions 222 that receive infrared and/or red light may be identified in the application running on computing device 110 and thus, according to various embodiments, may not be physical partitions but instead may be sections of the image captured/detected by the sensor 204. The resulting image may thus be analyzed to determine the light intensity of red light 221 and/or infrared light 219 detected at the partitions 222 of sensor 204 and the resulting images. The images may be a series of screen captures, a video image, and/or a still image. The application running on computing device 110 may analyze the resulting image to measure physiological/vital information, such as SpO₂ levels and heart rate.

Referring now to FIG. 3, a process 300 for analyzing images to determine levels/concentrations of various physiological compounds, such as SpO₂ levels and/or heart rate, is shown, in accordance with various embodiments. The application running on computing device 110 may execute process 300 against a video capture. The following terms and concepts, merely introduced in this paragraph, are utilized below to describe various steps or features of the process 300. Photoplethysmogram (PPG) signals are extracted from the video capture based on the intensity of the light (red, IR, or other suitable frequencies) detected. Extinction coefficients may influence the intensity of light captured in the image and calibration may correct for variance in the light intensity. The extinction coefficients may vary significantly due to the different sensors on different computing devices 110, placement of body part 214 on camera lenses, and incoming light source, for example. A calibration may tend to reduce error introduced by the above variables.

Captured signals 400 from the red channel (light at the red wavelength) and the infrared channel (light at the IR wavelength) are identified as signal 402 (red wavelength) and signal 404 (IR wavelength), respectively, and are shown in FIG. 4. Captured signals 400 of FIG. 4 may be the light intensity at the red wavelength and infrared wavelength as detected over the duration of a video image captured as sensor 204 of computing device 110. Heartrate may be determined from the image intensity of captured signals 400 by determining the frequency of one or more of the signals. Process 300 may include calculating, at each point “t” in the captured signals 400, the mean value at frame “t” of the channel as shown below with “t” ranging from 1 to end time T of the input video signal (Step 302). The calculation to determine Red Photoplethysmogram (“RPPG” or “rppg”) from the red channel and Infrared Photoplethysmogram (“IPPG” or “ippg”) from the infrared channel may use the below relationships.

rppg(t)=μ(lnpVid{r,t})

ippg(t)=μ(lnpVid{i,t})

The mean value of each frame “t” may be calculated by computing device 110 coupled to add-on device 104. Computing device 110 may then filter the signals using a bandpass filter which tends to allow only signals from 30 bpm (about 0.5 Hz) to 230 bpm (about 3.83 Hz) to pass (Step 304). The bandpass filter may be a Butterworth filter, for example, to have a flat frequency response in the pass band. Pulse oximetry techniques may adopt simple linear regression between absorptivity ratio and SpO₂, as SpO₂=−a*R+b. Referring to FIG. 5, the results from the image analysis of the red (rppg) and infrared (ippg) channels of FIG. 4 is shown, with a=24.87 and b=113.8, and with a 6 seconds window based approach.

Red channel 502 may have an “AC” and “DC” component, similar to those commonly described with reference to electrical signals. The AC component may be the waveform of increasing and decreasing values, and the DC component may be the offset from the value 0 to the lowest value of the AC component. Thus, the AC component of red channel 502 is the portion above DC-cutoff line 503, with the DC component being below DC-cutoff line 503. The DC component of red channel 502 is thus approximately 97.7, and the AC component of red channel 502 varies from about 99 to about 97.7. The AC and DC values of infrared channel 504 may be determined in a similar manner with reference to DC-cutoff line 505.

Computing device 110 may obtain the AC and DC values for light intensity of the red channel and the infrared channel as described above. Computing device 110 may then calculate the absorptivity ratio (R) using the below relationship (Step 306).

$$R = \frac{AC ( rppg ) \text{/} DC ( rppg )}{AC ( ippg ) \text{/} DC ( ippg )}$$

The extinction coefficients may be determined based on the relationship between wavelength and extinction coefficient by looking up the extinction coefficient using a wavelength. For example, for a red wavelength of approximately 620 nm, εHbO₂(620) is approximately 0.1, and εHb(620) is approximately 0.05.

Computing device 110 may then calculate SpO₂ levels (Step 308). Computing device 110 may calculate the SpO₂ levels using the below relationship.

$${SpO}_{2} = \frac{ɛ {HbO}_{2} ( 620 ) - ɛ Hb ( 850 ) R}{ɛ Hb ( 620 ) - ɛ {HbO}_{2} ( 620 ) + [ ɛ {HbO}_{2} ( 850 ) - ɛ Hb ( 85 ) ]}$$

The SpO₂ levels may be calculated at various times for a predetermined duration to generate plot 506 of SpO₂ levels. For example, SpO₂ levels may be calculated every second, every half second, for time intervals of a predetermined duration with the intervals overlapping, or over another suitable period. Computing device 110 may analyze resulting SpO₂ values (e.g., in plot 506) to identify undesirable SpO₂ levels. The resulting plot 506 and/or SpO₂ levels may be stored for long-term monitoring.

In various embodiments, a prediction error may be caused by the high fluctuation of absorption ratio and may be caused by the difference levels of red color between each finger. An absorption ratio may be calculated as follows:

$$R = \frac{rms ( Red ) - k * μ ( Red\_R )}{rms ( Green ) - k * μ ( Red\_G )}$$

The absorption ratio may be used to compensate the effect of red skin color variance by reducing the reflection of red light. In this formula, rms(Red), rms(Green) are the root mean square of Red and Green PPG signal, (Red_R), μ(Red_G) are the mean value of red color channel in red and green region respectively and k is the control variable to adjust the level of compensation.

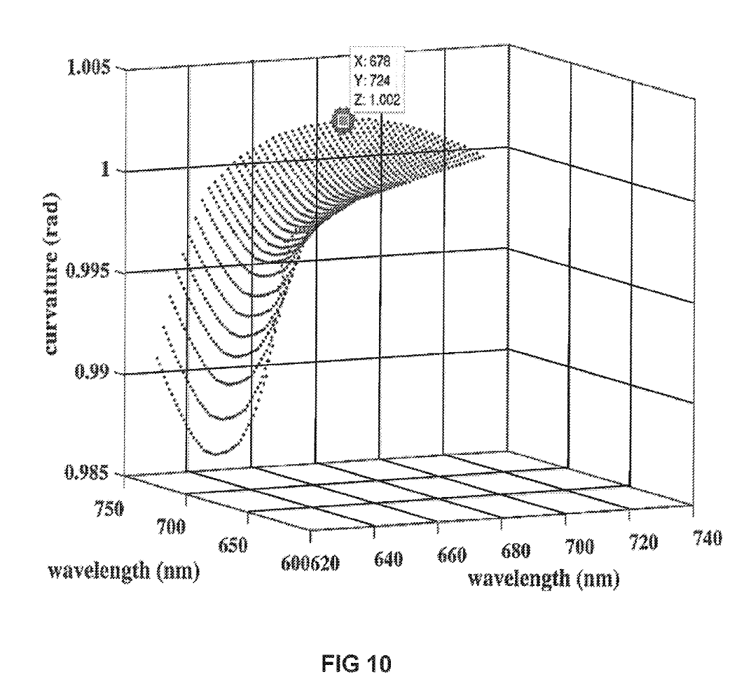

In various embodiments, and with reference to FIG. 10, determining the physiological information may involve a protocol of selecting certain wavelengths based on their level of ambiguity in the measurement of a specific physiological parameter, such as oxygen saturation. In various embodiments, for example, a correlation between SpO₂ and absorptivity ratio R with respects to different combinations of light source indicates that the curve of the relational curve between SpO₂ and R can help to identify the wavelengths. For example, electromagnetic radiation having wavelengths from 600 nm to 750 nm are used to calculate the curvature level of angle between the relational curve and the vertical axis. As shown in FIG. 10, the combination of light at 678 nm and 724 nm has the highest curvature at 1.002 radian. Such may be selected as the possible candidates and the method may further include searching for adjacent points (e.g., neighbors) to identify the optimal pair.

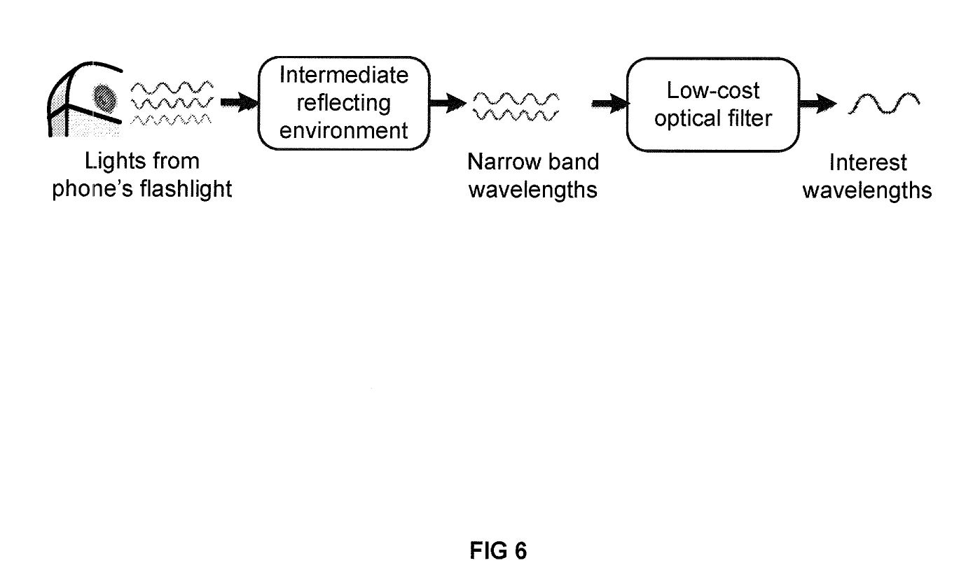

In various embodiments, and with reference to FIGS. 1A, 1B, and 6, the add-on device 104, which may also be referred to as a physiological information device, generally forms an intermediate reflecting environment between the light source, such as flash 108, and sensor, such as camera 106. That is, the add-on device 104 may include one or more lenses and one or more optical filters that are generally configured to control which wavelengths of light are utilized in the determination of the physiological information. In various embodiments, for example, the intermediate environment of the add-on device 104 is configured to absorb part of the light coming from the phone's flashlight and reflect the narrower band of wavelengths.

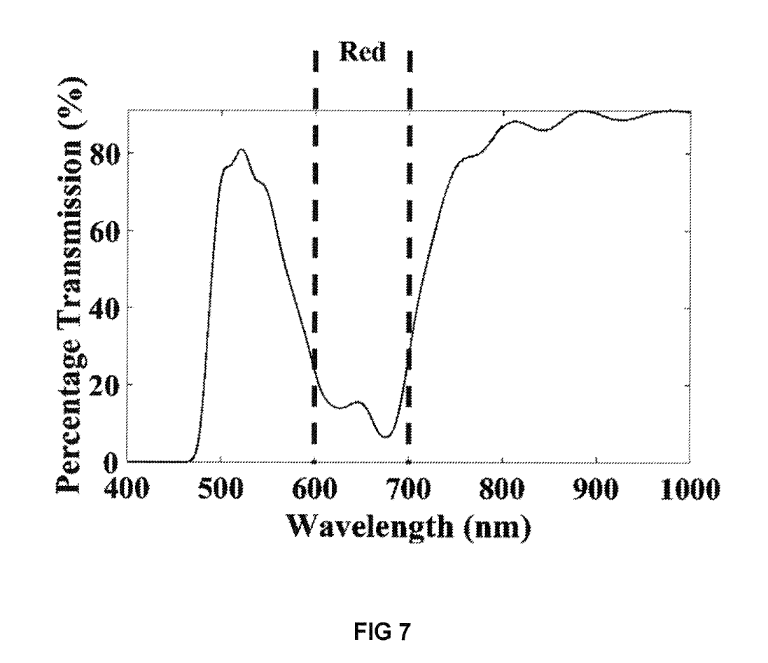

In various embodiments, an optical filter may be utilized to remove any remaining unwanted wavelengths of light, thereby leaving only the desired wavelengths. For example, in various embodiments, and with reference to FIG. 7, near-infrared (“NIR”) may be isolated from the light source. In various embodiments, red skin pigmentation of a body part (e.g., a finger) of a user plays a role in configuring the intermediate environment that absorbs the light. For example, the band of light from a conventional phone or tablet may be limited between about 400 nm to about 779 nm, including NIR and part of IR. Therefore, the reflected lights from the body part of the user may have the band of wavelength from 600 nm to 779 nm. Since infrared is blocked by most smartphones due to its negative effect on image quality, a green optical polyester filter may be positioned in the light pathway to eliminate the red components and capture only the NIR, as shown generally in FIG. 7.

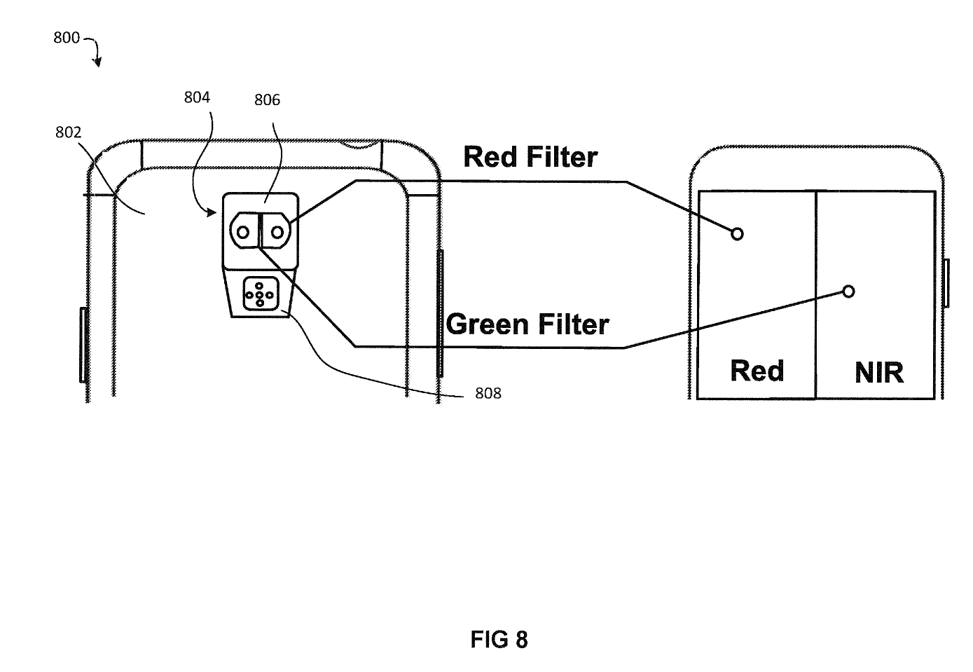

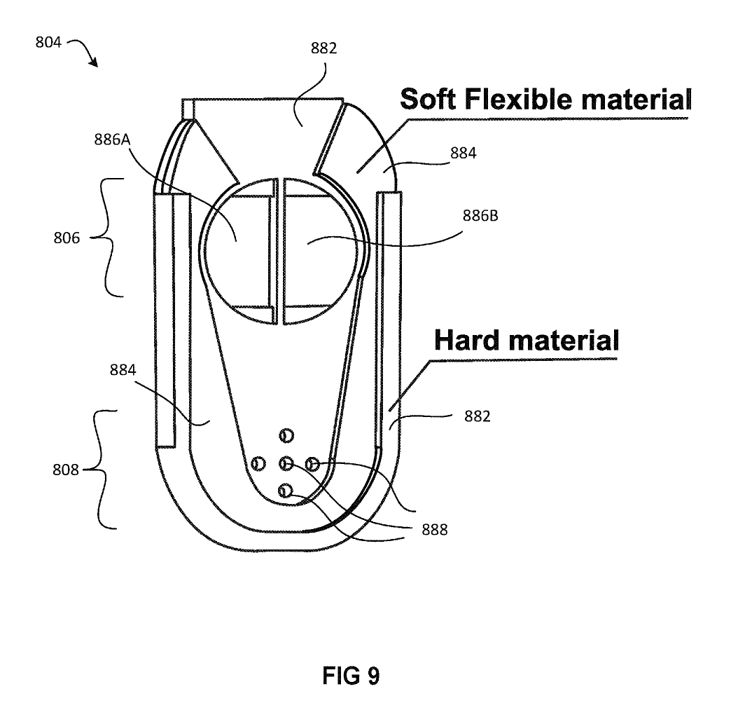

In various embodiments, and with reference to FIGS. 8 and 9, system 800 includes computing device 802 and physiological information device 804. As mentioned above, the physiological information device 804 may be an add-on device that is embedded/incorporated into a protective or decorative case for the computing device 802 or the physiological information device 804 may be independently attached, mounted, affixed, or otherwise coupled to the computing device 802. In various embodiments, the physiological information device 804 is generally configured to reflect, filter, and otherwise condition the light from the light source 108 (FIG. 1A) before it is captured/analyzed at camera 106 (FIG. 1A). In various embodiments, the body of the physiological information device 804 includes a camera covering portion 806 and a light covering portion 808. The light covering portion 808 may generally include the features and components (e.g., lenses and filters) shown on the left side of FIG. 2 while the camera covering portion 806 may generally include the features and components (e.g., lenses and filters) shown on the right side of FIG. 2. The camera covering portion 806 may include multiple filters. For example, the camera covering portion 806 may include a first filter 886A (e.g., red filter) configured to be disposed over a first half of the camera and a second filter 886B (e.g., green filter) configured to be disposed over a second half of the camera. The red and green filters may be used to obtain the image of red and NIR wavelengths, which may be analyzed in the computing device 802 and/or simultaneously displayed on the screen of the device 802 (as shown in the right half of FIG. 8).

In various embodiments, and with continued reference to FIG. 9, the light covering portion 808 may include one or more apertures 888 that can be used to condition (e.g., decrease) the light energy. In various embodiments, the build-up of heat is proportional to the light intensity of the light source. Thus, the light covering portion 808 may include multiple apertures that allow light to pass there through but dissipates heat by limiting the amount of light that enters flows into the body part and ultimately into the camera. In various embodiments, the radius of the apertures 888 is between about 0.6 mm to about 0.3 mm. For example, the light covering portion 808 may include five apertures 888, each with a radius of 0.3 mm.

In various embodiments, and with continued reference to FIG. 9, the body of add-on physiological information device 804 may be made from different materials to reduce the vibration and movement caused by the human's finger. For example, the device 804 may include a flexible, softer material 884 that has the ability to damp vibrational movements or any other mechanical disturbance to facilitate the stability of the PPG signals. For example, the softer material 884 may include a rubber material can deform and can absorb force. In the event that the computing device (e.g., phone) is moving, the soft material 884 may help to reduce or eliminate the cardio-ballistic artifact in the detected signal. Additionally, the soft material 884 may help to absorb the pressure caused by the body part (e.g., human finger) pressed against the device 804. The body of the device 804 may also include a more rigid, hard material 882 to provide structure to the device.

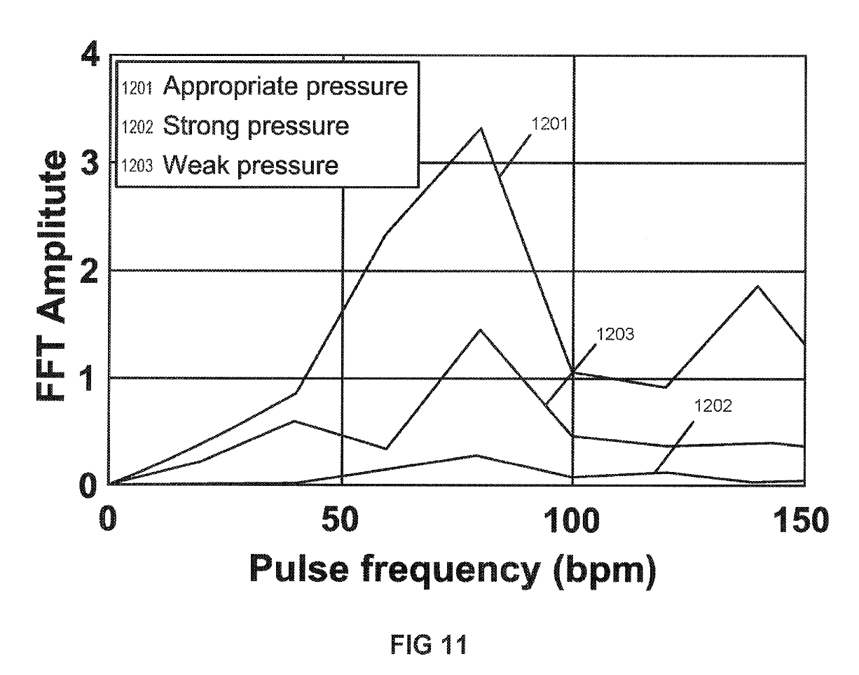

In various embodiments, and with reference to FIG. 11, the system may perform a pressure-based correction or may provide pressure-based feedback. That is, the system and device may be configured to manage measurement of physiological information despite the varying levels of pressure a user may exert when positioning a body part (e.g., a finger) against the device. In various embodiments, for example, a vision-based pressure detection using the Fast Fourier Transform (“FFT”) of PPG signal. For example, every “N” seconds of recorded video (e.g., 30 N frames), the mean intensity is processed and extracted in the red channel of each frame. The fluctuation or peak-to-peak variation of this sequence may have a large difference. Because this sequence also represents part of the PPG signal, the FFT is calculated with respected to heart beat frequency, as shown in FIG. 11, improved detection precision and accurate analysis decisions can result. For example, a user's force/press behavior may be classified into three categories: (1) strong press, in which intensity sequence is increased, no pulse is observed from recorded video; (2) weak press, in which intensity sequence is reduced, pulse is barely observed; and (3) normal/appropriate press, in which pulse is clearly observed, PPG signal is extracted without abnormalities. Therefore, the FFT feature of PPG signal may be used to identify the level of pressure the user is exerting on the device. In various embodiments, audible, visual, or other type of feedback may be provided to the user via the computing device to indicate to the user to press harder, softer, or maintain the current pressure.

In an example embodiment, the filters are in the case surrounding the device. For example, the filters are in the phone case. In an example embodiment, the light is in the case surrounding the device. For example, the light may be in the phone case. In an example embodiment, the camera is in the case surrounding the device. For example, the camera may be in the phone case. In an example embodiment, the processor is in the case surrounding the device. For example, the processor may be in the phone case. In this embodiment, the processor may communicate via Bluetooth or otherwise with the phone or other nearby devices. In an example embodiment, the device does not use the proximity sensor to perform the computations described herein. In an example embodiment, the device described herein can be used to compute: oxygen saturation, pulse rate, perfusion index, photoplethysmogram, blood pressure index, blood hydration, and/or body fat content; using the system described herein.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

Although the disclosure includes a method, it is contemplated that it may be embodied as computer program instructions on a tangible computer-readable carrier, such as a magnetic or optical memory or a magnetic or optical disk. All structural, chemical, and functional equivalents to the elements of the above-described various embodiments that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims.

Any reference to attached, fixed, connected or the like may include permanent, removable, temporary, partial, full and/or any other possible attachment option. Additionally, any reference to without contact (or similar phrases) may also include reduced contact or minimal contact. Surface shading lines may be used throughout the figures to denote different parts or areas but not necessarily to denote the same or different materials. In some cases, reference coordinates may be specific to each figure. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A physiological information system comprising:
a computing device comprising a light source and an image sensor; and
an add-on device in electronic communication with the computing device, wherein the add-on device comprises:
a body;
a first light filter retained in the body and configured to filter light emitted by the light source of the computing device;
a first lens retained in the body proximate the first light filter, wherein the first lens is configured to direct the light exiting the first light filter out a first surface of the first lens;
a second lens retained in the body and configured to receive into a second surface of the second lens the light exiting the first lens, wherein the first surface of the first lens and the second surface of the second lens are substantially coplanar; and
a second light filter retained in the body proximate the second lens, wherein the image sensor is configured to detect the light exiting the second light filter.

2. A method of detecting oxygen levels in blood using a computing device and an add-on device, comprising:
calculating a first mean value of light intensity detected by a first partition of an image sensor on the computing device at a first time for a red wavelength to generate a red PPG signal;
calculating a second mean value of light intensity detected by a second partition of the image sensor on the computing device at the first time for an infrared wavelength to generate an infrared PPG signal;
filtering the red PPG signal and the infrared PPG signal to allow frequencies ranging from 0.5 Hz to 4 Hz to pass;
calculating an absorptivity ratio from the red PPG signal and the infrared PPG signal; and
calculating the oxygen levels based on the absorptivity ratio.

3. A physiological information detection device comprising:
a body;
a first light filter retained in the body and configured to filter light emitted by a light source;
a first lens retained in the body proximate the first light filter, wherein the first lens is configured to direct the light exiting the first light filter out a first surface of the first lens;
a second lens retained in the body and configured to receive into a second surface of the second lens the light exiting the first lens, wherein the first surface of the first lens and the second surface of the second lens are substantially coplanar; and
a second light filter retained in the body proximate the second lens, wherein an image sensor is configured to detect the light exiting the second light filter.

4. The physiological information device of claim 3, wherein the physiological information device comprises a camera covering portion, wherein the camera covering portion comprises the second lens and the second filter.

5. The physiological information device of claim 4, wherein the camera covering portion comprises the second light filter and a third light filter.

6. The physiological information device of claim 5, wherein the second light filter is a red filter and the third light filter is a green filter.

7. The physiological information device of claim 3, wherein the physiological information device comprises a light covering portion, wherein the light covering portion comprises the first light filter and the first lens.

8. The physiological information device of claim 7, wherein the light covering portion comprises a plurality of apertures that limit an amount of light energy from the light source.

9. The physiological information device of claim 3, wherein the body comprises a flexible material and a rigid material.

10. The physiological information device of claim 9, wherein the flexible material is configured to absorb vibrational movement.
